(19) United States
(12) Patent Application Publication
(10) Pub. No.: US 2006/0235282 A1
(43) Pub. Date: Oct. 19, 2006

(54) Methods for the treatment of mammals with abnormal glucose tolerance

(76) Inventors: Helen Wesolowski, Locust Grove, VA (US); Marjorie Timko, Locust Grove, VA (US)

Correspondence Address: FLESHNER & KIM, LLP, P.O. BOX 221200, CHANTILLY, VA, 20153, US

(21) Appl. No.: 11/377,179

(22) Filed: Mar. 17, 2006

Related U.S. Application Data
(60) Provisional application No. 60/662,368, filed on Mar. 17, 2005.

(51) Int. Cl. A61B 5/00; A61B 5/02; A61B 5/103

(52) U.S. Cl. 600/300; 600/595; 600/587; 600/485; 128/921

(57) ABSTRACT

Disclosed are methods for improving insulin sensitivity and/or glucose metabolism in individuals with abnormal glucose tolerance. Such methods may be used in treating conditions associated with abnormal glucose tolerance and metabolism in mammals, including insulin resistance, insulin resistant metabolic syndrome, impaired glucose tolerance, impaired fasting glucose, hypertension, hypercholestemia and diabetes, particularly Type 2 diabetes.

This application claims benefit of U.S. Provisional Application Ser. No. 60/662,368 filed Mar. 17, 2006, whose entire disclosure is incorporated herein by reference.

### FIELD OF THE INVENTION

The present invention relates to methods for improving insulin sensitivity and/or glucose metabolism in individuals with abnormal glucose tolerance. Such methods may be used in treating conditions associated with abnormal glucose tolerance and metabolism in mammals, including insulin resistance, insulin resistant metabolic syndrome, impaired glucose tolerance, impaired fasting glucose, hypertension, hypercholesterolemia and diabetes, particularly Type 2 diabetes.

### BACKGROUND OF THE INVENTION

Obesity has now reached epidemic proportions globally, with more than 1 billion adults overweight—and at least 300 million of them clinically obese—and is a major contributor to the global burden of chronic disease and disability. Increased consumption of more energy-dense, nutrient-poor foods with high levels of sugar and saturated fats, combined with reduced physical activity, have led to obesity rates that have risen three-fold or more since 1980 in some areas of North America, the United Kingdom, Eastern Europe, the Middle East, the Pacific Islands, Australasia and China.

Obesity and overweight pose a major risk for serious diet-related chronic diseases, including Type 2 diabetes, cardiovascular disease, hypertension and stroke, and certain forms of cancer. The health consequences range from increased risk of premature death, to serious chronic conditions that reduce the overall quality of life; of special concern is the increasing incidence of child obesity.

The rising epidemic of obesity and overweight reflects the profound changes in society and in behavioral patterns of communities over recent decades. While genetics may be important in determining a person's susceptibility to weight gain, energy balance is determined by calorie intake and physical activity. Thus, societal changes and worldwide nutrition transition tend to drive the obesity and overweight epidemic.

Economic growth, modernization, urbanization and globalization of food markets are just some of the forces that have been suggest as contributing to this epidemic. In particular, as incomes rise and populations become more urban, diets high in complex carbohydrates give way to more varied diets with a higher proportion of fats, saturated fats and sugars. At the same time, large shifts towards less physically demanding work have been observed worldwide. Moves towards less physical activity are also found in the increasing use of automated transport, technology in the home, and more passive leisure pursuits.

The prevalence of overweight and obesity is commonly assessed by using body mass index (BMI), defined as the weight in kilograms divided by the square of the height in meters (kg/m²). A BMI over 25 kg/m² is defined as overweight, and a BMI of over 30 kg/m² as obese. These markers provide common benchmarks for assessment, but the risks of disease in all populations can increase progressively from lower BMI levels.

Raised BMI is also thought to increase the risks of cancer of the breast, colon, prostrate, endometrium, kidney and gallbladder. Chronic overweight and obesity contribute significantly to osteoarthritis, a major cause of disability in adults. Although obesity should be considered a disease in its own right, it is also one of the key risk factors for other chronic diseases together with smoking, high blood pressure and high blood cholesterol. In the analyses carried out for World Health Report 2002, approximately 58% of diabetes and 21% of ischemic heart disease and 8-42% of certain cancers globally were attributable to a BMI above 21 kg/m².

Adult mean BMI levels of 22-23 kg/m² are found in Africa and Asia, while levels of 25-27 kg/m² are prevalent across North America, Europe, and in some Latin American, North African and Pacific Island countries. BMI increases amongst middle-aged elderly people, who are at the greatest risk of health complications. In countries undergoing nutrition transition, over nutrition often co-exists with undernutrition.

The distribution of BMI is shifting upwards in many populations. And recent studies have shown that people who were undernourished in early life and then become obese in adulthood, tend to develop conditions such as high blood pressure, heart disease and diabetes at an earlier age and in more severe form than those who were never undernourished.

Currently more than 1 billion adults are overweight—and at least 300 million of them are clinically obese. Current obesity levels range from below 5% in China, Japan and certain African nations, to over 75% in urban Samoa. But even in relatively low prevalence countries like China, rates are almost 20% in some cities.

Childhood obesity is already epidemic in some areas and on the rise in others. An estimated 22 million children under five are estimated to be overweight worldwide. According to the US Surgeon General, in the USA the number of overweight children has doubled and the number of overweight adolescents has trebled since 1980. The prevalence of obese children aged 6-11 years has more than doubled since the 1960s. Obesity prevalence in juveniles aged 12-17 has increased dramatically from 5% to 13% in boys and from 5% to 9% in girls between 1966-70 and 1988-91 in the USA. The problem is global and increasingly extends into the developing world; for example, in Thailand the prevalence of obesity in 5-to-12 year olds children rose froml2.2% to 15-6% in just two years.

Obesity accounts for 2-6% of total health care costs in several developed countries; some estimates put the figure as high as 7%. The true costs are undoubtedly much greater as not all obesity-related conditions are included in the calculations.

Overweight and obesity lead to adverse metabolic effects on blood pressure, cholesterol, triglycerides and insulin resistance. The non-fatal, but debilitating health problems associated with obesity include respiratory difficulties, chronic musculoskeletal problems, skin problems and infertility. The more life-threatening problems fall into four main areas: CVD problems; conditions associated with insulin resistance such as type 2 diabetes; certain types of cancers, especially the hormonally related and large-bowel cancers; and gallbladder disease.

The likelihood of developing Type 2 diabetes and hypertension rises steeply with increasing body fatness. Confined to older adults for most of the 20th century, this disease now affects obese children even before puberty. Approximately 85% of people with diabetes are type 2, and of these, 90% are obese or overweight. And this is increasingly becoming a developing world problem. In 1995, the Emerging Market Economies had the highest number of diabetics. If current trends continue, India and the Middle Eastern crescent will likely have taken over by 2025. Large increases would likely also be observed in China, Latin America and the Caribbean, and the rest of Asia.

Abnormal glucose tolerance refers to metabolic stages intermediary to normal glucose homeostasis and Type 2 diabetes; this includes conditions like impaired glucose tolerance (IGT) and impaired fasting glucose (JFG), where glucose values are above the conventional normal range and are often accompanied by a decrease in insulin sensitivity. Impaired glucose tolerance (IGT) and impaired fasting glucose (IFG) are transient, intermediate stages in the development of Type 2 diabetes. Within ten years of diagnosis, approximately 30% of IGT subjects will progress to Type 2 diabetes and potentially to health problems that accompany this disease, including retinopathy, nephropathy, and peripheral neuropathy. In addition, abnormal glucose tolerance and decreased insulin sensitivity are associated with a high risk for the development of hypertension, dyslipidemia and an increase incidence of coronary artery disease.

Abnormal glucose tolerance and decreased insulin sensitivity can be attributed to a wide range of causes including obesity, age, physical activity level, certain medication or drugs, genetic factors, and some endocrine related disorders. The truncal distribution of weight as determined by a high waist to hip ratio (WHR) is a good predictor of abnormal insulin sensitivity, and there is an excellent correlation between a high body mass index (BMI) and decreased insulin sensitivity. Approximately 33% of the population in the United States is obese, and the majority of these individuals have decreased insulin sensitivity, are hyperinsulinemic, and often have abnormal glucose tolerance.

Impaired fasting glucose (IFG) is defined by the American Diabetes Association as a fasting blood glucose in the range of 110 mg/dL to 125 mg/dL. IFG is determined by analysis of a plasma sample for glucose after a 10-16 hour fast. This is an easy and quick way to determine if there is a problem with glucose tolerance and metabolism.

Impaired glucose tolerance (JGT), as defined by the World Health Organization, is determined by the administration of a standard oral glucose tolerance test (OGT) (World Health Org., Diabetes Mellitus, Tech. Rep. Ser., no. 727 (1985)). During an OGTT, a measured amount of glucose (75 grams) is given to a fasted individual and blood glucose levels are measured every 30 minutes, usually for 2 or 3 hours. In a individual with normal glucose tolerance, the blood glucose values will rise during the first part of the test and then rapidly return to basal levels. In an IGT individual the post prandial glucose levels will rise above the normal range, producing a 2-hour glucose value of 140-199 mg/dL, and return to basal levels at a slow rate.

Abnormal glucose tolerance is caused in part by inadequate utilization of glucose in the periphery—at the site of the muscles. In addition, high fasting glucose values, seen with impaired fasting glucose, suggest that hepatic glucose production is not being effectively regulated. The underlying cause of this abnormal glucose tolerance is characterized by a decrease in insulin sensitivity.

Insulin sensitivity is a measurement of insulin's ability to produce a biological response; specifically, in the case of glucose regulation, insulin sensitivity is a measurement of insulin's ability to promote the clearance and utilization of glucose. A decrease in insulin sensitivity will result in a prolonged elevation of glucose levels and the release of additional insulin to try and achieve a euglycemic state. This compensatory hyperinsulinemia will effect insulin's ability to suppress lipolysis in adipose tissue, thus causing an increase in free fatty acids and ultimately resulting in the disruption of normal lipid profiles which could lead to coronary artery disease. The increase in free fatty acids will also inhibit insulin-stimulated glucose utilization in the muscle and stimulate hepatic gluconeogenesis. This leads to increased blood glucose and will eventually result in the development of impaired glucose tolerance or impaired fasting glucose and ultimately, if unchecked, the development of Type 2 diabetes. Improving insulin sensitivity will restore overall glucose control and decrease the risk of cardiovascular disease.

The level of insulin sensitivity can be measured to varying degrees by three methods: fasting plasma insulin, the euglycemic clamp, and the frequently sampled intravenous glucose tolerance test (FSIGTT). With the use of any of these techniques there is a wide range of insulin sensitivity, with overlapping values characterizing individuals with normal, abnormal glucose tolerance and Type 2 diabetes.

One method of determining insulin sensitivity is by fasting plasma insulin values. Simply, a high fasting insulin value correlates with decreased insulin sensitivity.

The euglycemic clamp test procedure involves infusing glucose at a variable rate in order to obtain a constant plasma glucose concentration. This rate of glucose infusion is equal to the overall rate of basal glucose disposal in the body. When insulin is also infused, the glucose infusion rate reflects insulin mediated glucose uptake. This is a precise and reproducible way to determine insulin sensitivity, albeit the euglycemic clamp method is time-consuming and complicated to perform.

A third method of determining insulin sensitivity involves frequently collecting blood samples for glucose and insulin during an intravenous glucose tolerance test, and analyzing the glucose and insulin dynamics using a minimal mathematical model developed by Bergman (Bergman et. al., J. Clin. Invest. 68:1456-1467 (1981)). This test can be modified by the injection of tolbutamide or exogenous insulin to boost the insulin response and improve the correlation with the euglycemic clamp. The FSIGTT provides an accessible way to determine the insulin sensitivity index (S₁). The more insulin insensitive a subject is, the lower the calculated S₁. As insulin sensitivity is improved, the S₁ value is increased—thus a higher S₁ value corresponds to greater insulin sensitivity.

Improving insulin sensitivity and glucose tolerance will help delay and even prevent the onset of Type 2 diabetes mellitus, and thus improve the quality of life by preventing acute and long-term complications, reducing mortality and treating accompanying disorders of those at risk for Type 2 diabetes.

Accordingly, there remains a need in the art for methods of improving insulin sensitivity and restoring normal glucose tolerance and metabolism in individuals with impaired glucose tolerance, impaired fasting glucose and/or Type 2 diabetes mellitus.

### SUMMARY OF THE INVENTION

The invention relates to methods for selecting a diet and exercise regimen for a patient and to methods for treating a patient comprising selecting a diet and exercise regimen.

In one embodiment, the invention provides a method for selecting a diet and exercise regimen for a patient, comprising conducting a particular physical therapy evaluation and a particular nutritional education evaluation of the patient; and selecting a diet and exercise regimen for the patient based on the results of those two evaluations.

In yet a further embodiment, the invention provides a method for treating a patient, comprising selecting a diet and exercise regimen according to the method described above; prescribing the selected diet and exercise regimen to the patient; monitoring the results achieved by the patient; and modifying the diet and/or exercise regimen based on those results.

### BRIEF DESCRIPTION OF THE DRAWINGS

FIG. 1 shows a Nutrition Education and Physical Therapy Floxy Chart that may be used in conjunction with the methods of the present invention disclosed herein.

FIG. 2 shows a Screening Protocol for School Children that may be used to identify patients who might benefit from the methods of the present invention disclosed herein.

FIG. 3 shows results achieved using the methods of the present invention.

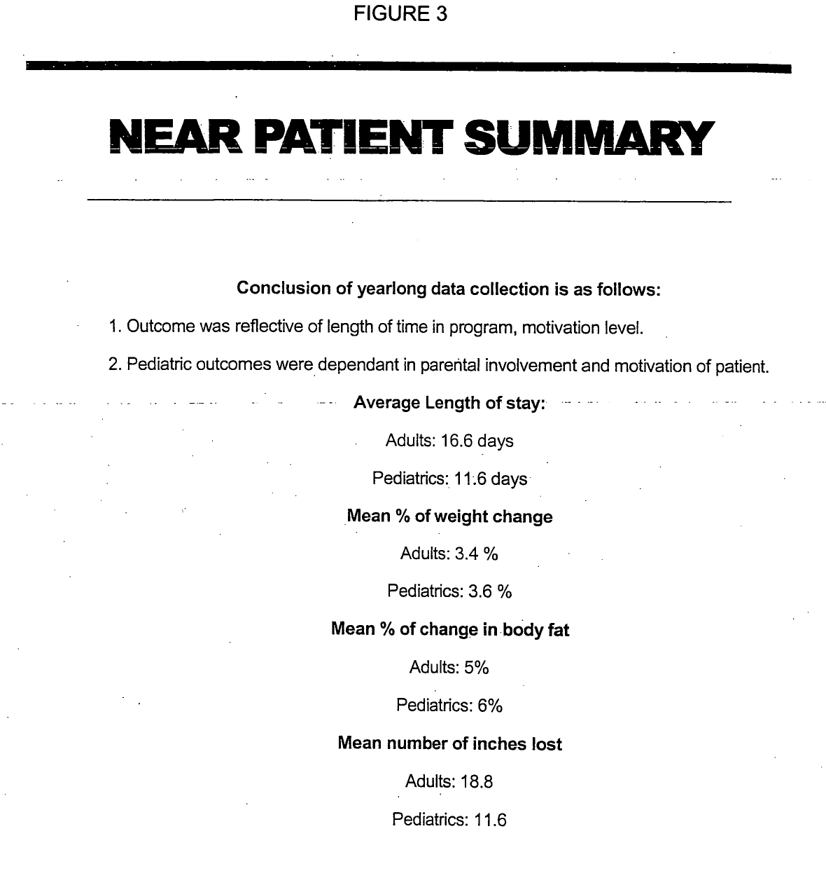

### DETAILED DESCRIPTION OF THE INVENTION

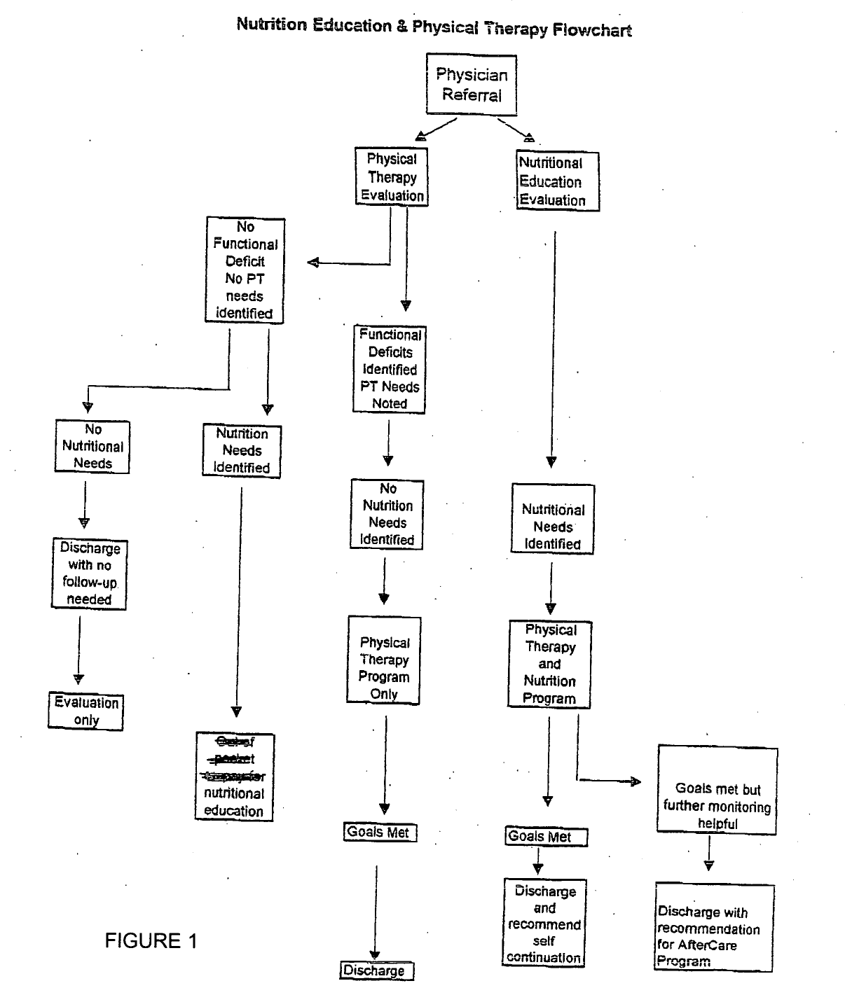

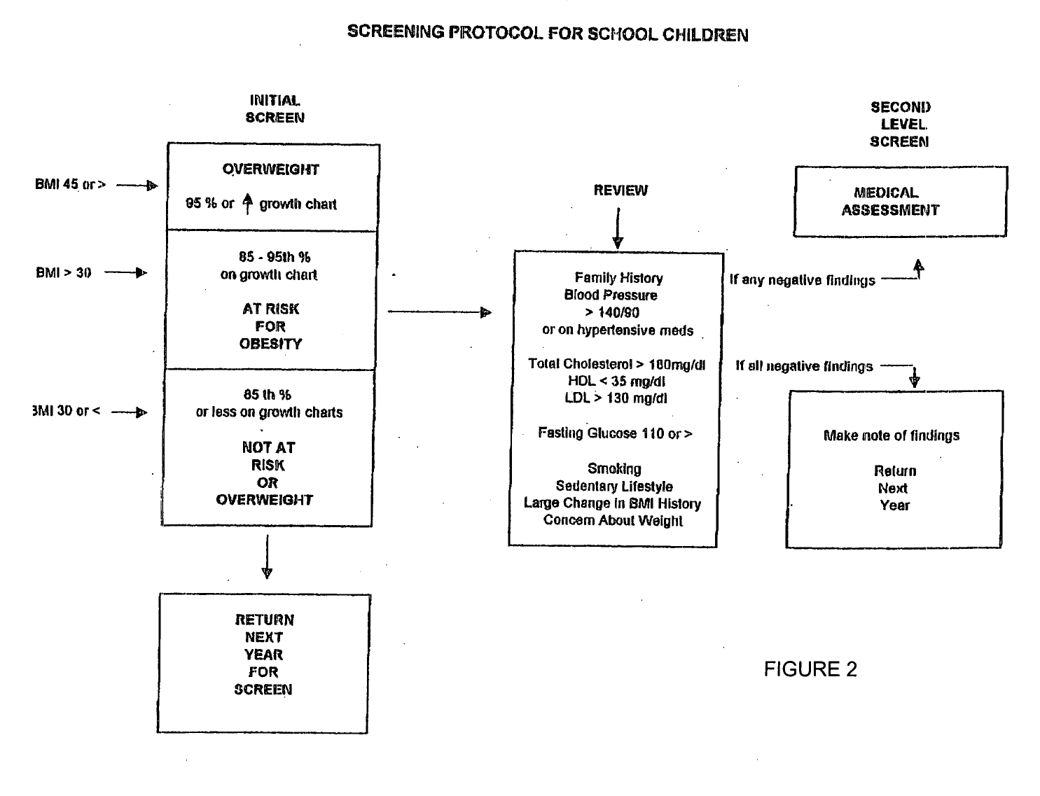

The invention includes methods for selecting an improved or optimal diet and exercise regimen for a patient based on the consideration of several factors, both physical and nutritional. FIG. 1 shows a flowchart for evaluating a general patient on first visit. FIG. 2 shows a screening protocol for evaluating a juvenile patient for possible type 2 diabetes or a precursor condition thereof, such as IFG, IGT and/or insulin resistant metabolic syndrome.

In one aspect of the present invention, a physical therapy evaluation of the patient is performed at the outset of treatment in order to establish “baseline” standards for the various data points. Based, at least in part of the results of that evaluation, a particularized exercise regiment is devised for that patient.

An initial physical therapy evaluation should cover a wide range of potential causes for concern during treatment, including, but not necessarily limited to, the following: (i) pain issues/concerns; (ii) range of motion; (iii) flexibility; (iv) muscle strength; (v) muscle endurance; (vi) sensory issues; (vii) functional deficits; and (viii) proprioception/balance/fall risk/coordination assessment and cardio-respiratory level.

Periodically during the course of treatment under the inventive method, the patient will be subject to further such evaluations as described above. Such follow-up evaluations allow the particular exercise regimen for that patient to be adjusted and controlled, as well as allowing the caregiver to monitor the patient's progress towards his/her goals in the program and to adjust the exercise regiment accordingly.

A suitable physical therapy evaluation protocol is shown below:

[image]

[image]

The use of the above Fitness Performance Measurement Grid is particularly advantageous with the methods of the present invention. Such a grid may be used during the initial Physical Therapy Evaluation (to establish baseline values) and throughout the period of treatment, either continuously or periodically, to monitor the results achieved with a given patient and suggest modifications and additions to the exercise regimen.

Additional factors which may be considered in conjunction with the physical therapy evaluation, particularly based on the Fitness Performance Measurement Grid, include, but are not limited to, the following: blood pressure, both at rest and with physical activity; heart rate, both at rest and with physical activity; blood sugar level and reaction to exercise/activity; patient's knowledge bases of diabetes and/or blood sugar and the relation to exercise/activity; psycho-social issue.

In is advantageous in certain embodiments of the invention to ascertain detailed physical data regarding a patient's body composition, for example by taking weight and size measurements as certain points of the body, for example abdominal thickness and skin fold caliper results. A suitable individual weight and measurement evaluation is shown below:

[image]

In addition to the above physical therapy evaluation and individual weight and measurement, a nutritional education evaluation is also advantageously conducted. Such an evaluation should, at least, be conducted at the outset of treatment to establish “baseline” values for future use and consideration. Preferably, a nutritional evaluation is conducted periodically during the treatment phase and the patient's suggested diet adjusted accordingly.

Such a nutritional education evaluation includes consideration of:

(i) Diet History

(a) assess for intake adequacy;

(b) review for vitamin and/or mineral deficiencies;

(c) review for caloric and/or carbohydrate abuse;

(d) review for food group avoidance;

(e) evaluate possible of eating disorder, e.g. anorexia, bulimia; and

(f) review current medications

(ii) Medical History

(a) risks of co-morbidity, e.g. sleep apnea, elevated cholesterol, hypertension, ADHD;

(b) abnormal laboratory values in body fluids (blood, urine, etc);

(c) possible food/drug interactions—dietary precautions;

(d) biometrics/anthropometrics;

(e) basal energy expenditure; and

(f) percentile placement on growth chart;

(iii) Social Implications

(a) family medical history (particularly diabetes, hypertension, hypercholesterolemia, coronary artery disease, alcoholism, depression, drug abuse, smoking, gestational diabetes);

(b) eating habits particularly fast food, skip meal, frequent eating out); and

(c) if juvenile, child's history (particularly latch-key, school breakfast and/or lunch programs, etc).

(iv) Socioeconomic

(a) number of family members;

(b) food stamps; and

(c) low or fixed income.

(v) Motivation Level/Education Limitations

(a) reading difficulty;

(b) comprehension challenges;

(c) visual learner; and

(d) inability to read/write.

Consideration of a plurality of the above factors from each of the physical therapy evaluation, the individual weight and measurement, and the nutritional educational evaluation, and particularly of those factors in combination, may advantageously be used in selecting an improved or optimal diet and exercise regimen according to the methods of the invention for a particular patient.

More specifically, the results from the physical therapy and nutritional education evaluations provide the caregiver with the ability to develop a baseline determination of a particular patient's risk profile. This determination is the first step in changing the management of the patient's disease. The accurate determination of the patient's risk factors will guide the development of a personalized treatment plan based on physical activity and diet. Such as individual's treatment plan can then be modified over time according to the results of follow-up testing.

Moreover, the invention further comprises methods for treating a patient comprising selecting a diet and exercise regimen based on the above-described method of selecting a diet and exercise regimen, monitoring the patient throughout the treatment and modifying the diet and/or exercise regimen as appropriate.

Patients with elevated glucose levels (e.g., 110 mg/dL or higher) should be monitored closely in order to prevent further disease progression and additive complications such as Type-2 diabetes (Packard C et al. (2000) Int J Cardiol 74 Suppl 1:S17-22). Calorie content of preventive diet must be appropriate for weight maintenance or weight loss depending on calculated goal weight or % body fat. Diet should be high in soluble fiber (oat bran, fruits, vegetables), whole grains/starches, fish and plant (soy) proteins and low in processed grains/starches (no sucrose or fructose) and animal proteins. Supplementation with Omega-3 fatty acids (fish oil, flax seeds) is suggested (inihane A M et al. (2000) Arterioscler Thromb Vasc Biol 20:1990-7). A smaller amount of food at more frequent intervals is also recommended as increasing meal frequency (but not total caloric intake) decreases cardiovascular risk. Simple sugars should be avoided. Diet should include fish at least twice each week or should be supplemented with fish oils or flax seeds. Low glycemic index foods should be chosen. Alcohol should be avoided. Careful control of caloric intake is suggested. Patients should refer to “low”, “moderate” and “high” food from the Glycemic Index Table.

Patients with elevated insulin levels (e.g., 12 uU/ml or higher) should be monitored closely in order to prevent further disease progression and additive complications such as Type-2 diabetes. Calorie content of preventive diet must be appropriate for weight maintenance or weight loss depending on calculated goal weight or % body fat. Diet should be high in soluble fiber (oat bran, fruits, vegetables), whole grains/starches, fish and plant (soy) proteins and low in processed grains/starches (no sucrose or fructose) and animal proteins. Supplementation with Omega-3 fatty acids (fish oil, flax seed) is suggested. A smaller amount of food at more frequent intervals is also recommended as increasing meal frequency (but not total caloric intake) decreases cardiovascular risk. Simple sugars should be avoided. Diet should include fish at least twice each week or should be supplemented with fish oils or flax seeds. Low glycemic index foods should be chosen. Alcohol should be avoided. Careful control of caloric intake is suggested. Patients should refer to “low”, “moderate” and “high” food from the Glycemic Index Table.

The population of patients with diabetes mellitus represents a very large percentage of patients with high cardiovascular risk (Superko H R (1999) Curr Atheroscler Rep 1:50-7). It is very likely that traditional treatment of these patients actually worsened their condition. Calorie content of preventive diet must be appropriate for weight maintenance or weight loss depending on calculated goal weight or % body fat. Diet should be high in soluble fiber (oat bran, fruits, vegetables), whole grains/starches, fish and plant (soy) proteins and low in processed grains/starches (no sucrose or fructose) and animal proteins. Supplementation with Omega-3 fatty acids (fish oil, flax seed) is suggested. A smaller amount of food at more frequent intervals is also recommended as increasing meal frequency (but not total caloric intake) decreases cardiovascular risk. Simple sugars should be avoided. Diet should include fish at least twice each week or should be supplemented with fish oils or flax seeds. Low glycemic index foods should be chosen. Alcohol should be avoided. Smaller meals and more frequent feeding are recommended. Careful control of caloric intake is suggested. Patients should refer to “low”, “moderate” and “high” food from the Glycemic Index Table.

The following examples are presented for illustrative purposes only and are not intended, nor should they be construed, as limiting the invention in any way. Those skilled in the art will recognize that variations on the following can be made without exceeding the spirit or scope of the invention.

Foods for use in the diet regimen of the invention can be divided into food categories and subcategories as “healthy, not-as-healthy and splurge” foods. The following list provides examples.

GRAIN & STARCH “healthy” foods are those that are good sources of soluble fiber.

VEGETABLE “healthy” foods are the most nutrient packed vegetables having three or more of Vitamin E, A, C, K or that are high in soluble fiber.

PROTEIN “healthy” designates those foods that are categorized by the food exchange system as very lean and includes salmon and soy products because of their heart-healthy properties. The “not-as-healthy” category includes meats/meat-substitutes categorized as lean and the “splurge” foods as medium to high-fat.

DAIRY nonfat and soy (again for its heart-healthy properties) products are listed as “healthy” whereas higher fat choices are in the other two categories.

FAT categories are divided relative to the type of major fatty-acid the food supplies: “healthy” are those fats that are primarily made up of monounsaturated fatty acids, or omega-3-fatty acids or that are high in isoflavones, “not-as-healthy” are fats containing mostly polyunsaturated fatty acids and “splurge” are those fats that are made up primarily of saturated fatty acids.

SWEETS categories are divided relative to simple sugar content and dietary fiber.

Physical activity can positively influence plasma lipid and lipoprotein concentration and reduce coronary artery disease risk levels (Caso E K (1950) Journal of the American Diabetic Association 26:575-583). In summary, physical activity and weight loss have been shown to decrease VLDL, IDL, LDL, APO B, triglycerides, HDL 3, insulin, blood glucose, fibrinogen levels as well as reverse non-insulin dependent diabetes, improve LDL subclass distribution, fibrinolysis and blood pressure and increase LDL particle size, insulin sensitivity and HDL 2 concentrations (Durstine J L and Thompson P D (2001) Cardiol Clin 19:471-88; Haskell W L (1994) Med Sci Sports Exerc 26:649-660).

The appropriate amount of exercise to promote these responses is dependent upon gender, age, height relative to weight, and the combined risk level of each individual (Halle M et al. (1999) Int J Sports Med 20:464-9). The appropriate duration for most activity is around 20-30 minutes but this can vary depending on the intensity of the activity. For reducing cardiovascular risk levels, it is more important that physical activity is performed on a regular basis as the short-term (acute) effects of exercise that last up to 72 hours greatly improve patients' metabolic condition. Long-term exercise training increases exercise capacity, which permits larger individual exercise sessions and a greater acute effect. The exercise goal to work towards is 20-30 minutes of continuous physical activity on most days of the week.

The methods of the invention quantify and qualify how much physical activity and the type of such physical activity is appropriate to reduce risk factors relative to an individual's personal set of demographic, medical, and other risk parameters.

[image]

[image]

[image]

[image]

[image]

[image]

[image]

[image]

[image]

[image]

[image]

[image]

[image]

[image]

[image]

[image]

[image]

[image]

[image]

All of the articles, books, patents, patent applications, and other references cited in this patent application are hereby incorporated by reference.

Although certain presently preferred embodiments of the invention have been described herein, it will be apparent to those of skill in the art to which the invention pertains that variations and modifications of the described embodiment may be made without departing from the spirit and scope of the invention. Accordingly, it is intended that the invention be limited only to the extent required by the following claims and the applicable rules of law.

What is claimed is:

1. A method for determining a diet and exercise regimen for a patient in need thereof, comprising conducting a particular physical therapy evaluation and a particular nutritional education evaluation of the patient; and selecting a diet and exercise regimen for the patient based on the results of those two evaluations.

2. The method according to claim 1, wherein said physical therapy evaluation comprises: (a) a range of motion/flexibility evaluation; (b) a muscle strength/endurance evaluation; and (c) a cardio-respiratory fitness evaluation.

3. The method according to claim 2, wherein said physical therapy evaluation further comprises (d) a body composition evaluation.

4. The method according to claim 2, wherein said range of motion/flexibility evaluation comprises determining range of motion/flexibility for one or more of: (a) sit and reach; (b) pectorals; (c) hamstrings; (d) tensor fascia latae; and (e) hip flexors.

5. The method according to claim 2, wherein said muscle strength/endurance evaluation comprises determining one or more of: (a) left strength; (b) right grip strength; (c) number of sit-ups in 60 seconds; (d) maximum number of push-ups; (e) one repetition maximum bench press; and (f) one repetition maximum leg press.

6. The method according to claim 2, wherein said cardio-respiratory fitness evaluation comprises walking on a treadmill for 3 minutes at one or more of: (a) 1.5 mph, no incline; (b) 1.7 mph, 5 degrees incline; (c) 2.5 mph, 7 degrees incline; (d) 3.0 mph, 9 degrees incline; and (e) 1.7 mph, 0 degrees incline.

7. The method according to claim 2, wherein said physical therapy evaluation further comprises determining one or more of said patient's: resting heart rate; heart rate with physical activity; blood sugar level testing; blood sugar level reaction to physical activity; resting blood pressure; and blood pressure with physical activity.

8. The method according to claim 3, wherein said body composition evaluation comprises determining one of more of said patient's: height; weight; neck diameter; shoulder width; chest diameter; abdomen diameter; waist diameter; hip diameter; left bicep diameter; right bicep diameter; left forearm diameter; and right forearm diameter.

9. The method according to claim 8, wherein said body composition evaluation further comprises skin fold measurements at one or more of said patient's: left thigh; left knee; left calf; right thigh; right knee; and right calf.

10. The method according to claim 2, wherein said nutritional education evaluation comprises consideration and evaluation of one or more of: (a) diet history; (b) medical history; (c) social implications; (d) socioeconomic factors; and (e) motivation level/education limitations.

11. The method according to claim 10, wherein said consideration and evaluation of diet history comprises assessing one or more of: (a) intake adequacy; (b) vitamin and/or mineral deficiencies; (c) caloric and/or carbohydrate abuse; (d) food group avoidance; (e) possibility of eating disorder; and (f) effect of current medications.

12. The method according to claim 10, wherein said consideration and evaluation of medical history comprises assessing one or more of: (a) risks of co-morbidity; (b) abnormal laboratory values in body fluids; (c) possible food/drug interactions; (d) biometrics/anthropometrics; (e) basal energy expenditure; and (f) percentile placement on growth chart.

13. The method according to claim 10, wherein said consideration and evaluation of social implications comprises assessing one or more of the following: (a) family medical history; (b) eating habits; and (c) if said patient is juvenile, said patient's personal circumstances for presence of latch-key, school breakfast and/or lunch programs.

14. The method according to claim 13, wherein said assessing of family medical history comprises reviewing for the presence of one or more of: diabetes; hypertension; hypercholesterolemia; coronary artery disease; alcoholism; depression; drug abuse; smoking; and/or gestational diabetes.

15. The method according to claim 10, wherein said consideration and evaluation of socioeconomic factors comprises assessing one or more of the following: (a) number of family members; (b) food stamps; and (c) low or fixed income.

16. The method according to claim 10, wherein said consideration and evaluation of motivation level/education limitations comprises assessing one or more of the following: (a) reading difficulty; (b) comprehension challenges; (c) visual learner; and (d) inability to read/write.

17. A method for treating a patient suffering from conditions associated with abnormal glucose tolerance and metabolism, comprising selecting a diet and exercise regimen according to the method of claim 1; prescribing the selected diet and exercise regimen to the patient; monitoring the results achieved by the patient; and modifying the diet and/or exercise regimen based on those results.

18. The method according to claim 17, wherein said patient is suffering from insulin resistance, insulin resistant metabolic syndrome, impaired glucose tolerance, impaired fasting glucose, hypertension, hypercholestemia and/or diabetes.

19. The method according to claim 17, wherein said patient has a blood glucose level of 110 mg/dl or higher.

20. The method according to claim 17, wherein said patient has an insulin level of 12 μU/ml or higher.
